(19) United States
(12) Patent Application Publication
(10) Pub. No.: US 2024/0337066 A1
(43) Pub. Date: Oct. 10, 2024

(54) COMPOSITION AND METHOD FOR ODOR REDUCTION AND BACTERIAL CONTROL ON A TEXTILE

(71) Applicant: Microban Products Company, Huntersville, NC (US)

(72) Inventors: Yihong LI, Huntersville, NC (US); Ivan Wei Kang Ong, Charlotte, NC (US); Xiuzhu Fei, Charlotte, NC (US)

(21) Appl. No.: 18/747,927

(22) Filed: Jun. 19, 2024

Related U.S. Application Data
(62) Division of application No. 17/845,924, filed on Jun. 21, 2022.
(60) Provisional application No. 63/216,321, filed on Jun. 29, 2021.

(51) Int. Cl. D06M 13/203; D06B 3/10; D06B 3/18; D06M 16/00

(52) U.S. Cl. CPC D06M 13/2035; D06B 3/10; D06B 3/18; D06M 16/00

(57) ABSTRACT

A method for reducing microbial growth and odor on a textile material are provided. The method comprises applying a composition to a textile material. The composition consists of a salt of benzoic acid; a non-benzoic acid consisting of sorbic acid, citric acid, lactic acid, acetic acid, L-glutamic acid, trisodium citrate, potassium sorbate, and combinations thereof. The composition has a weight ratio of salt of benzoic acid to non-benzoic acid ranging from 1:3 to 10:1 and the composition reduces microbial growth on the textile after laundering.

### FIELD OF THE INVENTION

The present invention relates to a composition and method for odor control, more particularly to a composition and method for durable odor control on a textile.

### BACKGROUND OF THE INVENTION

Current bacterial control on textile garments relies heavily on the use of metal containing actives, such as zinc. These “heavy metal” presences in process effluent water in textiles mills has been an issue of concern for environmental policy and regulators. It will ultimately result in an inability to utilize zinc pyrithione to reduce odor and stain causing bacteria on textile surfaces.

Alternatively, the use of organic molecules, such as quaternary ammonium silanes, phenolics, and azoles, can be leveraged to treat textile surfaces. These chemistries require extensive formulary efforts to reduce hydrophobicity, increase durability.

This invention provides an antimicrobial solution for textile substrates which meets a new regulatory requirement, does not impart hydrophobicity, and provides good durability.

### SUMMARY OF THE INVENTION

The present invention relates to a composition and method for durable odor control on a textile.

In an embodiment of the invention, a composition is provided. The composition comprises: benzoic acid, a salt of benzoic acid, non-benzoic acid, or a combination of the non-benzoic acid and the salt of non-benzoic acid; and an optional binder.

In an embodiment of the invention, a composition is provided. The composition comprises: sodium benzoate, and citric acid.

In an embodiment of the invention, a composition is provided. The composition comprises: a component selected from the group consisting of benzoic acid, a salt of benzoic acid, and a combination thereof; and a binder.

In an embodiment of the invention, a method is provided. The method comprises mixing benzoic acid and/or a salt of the benzoic acid, and an optional binder. The method may further comprise exhausting a textile using the mixture in an exhaustion bath.

In an embodiment of the invention, a method is provided. The method comprises mixing sodium benzoate with a non-benzoic acid and/or a salt of the non-benzoic acid, and an optional binder. The method may further comprise exhausting a textile using the mixture in an exhaustion bath.

In an embodiment of the invention, a method is provided. The method comprises mixing benzoic acid and/or a salt of the benzoic acid, and an optional binder. The method may further comprise padding the mixture on a textile.

In an embodiment of the invention, a method is provided. The method comprises mixing sodium benzoate with a non-benzoic acid and/or a salt of the non-benzoic acid, and an optional binder. The method may further comprise padding the mixture on a textile.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### DESCRIPTION OF THE PREFERRED EMBODIMENTS

The following description of the embodiments of the present invention is merely exemplary in nature and is in no way intended to limit the invention, its application, or uses. The following description is provided herein solely by way of example for purposes of providing an enabling disclosure of the invention, but does not limit the scope or substance of the invention.

Further, the term “or” as used in this disclosure and the appended claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in,” “at,” and/or “on,” unless the context clearly indicates otherwise. The phrase “in one embodiment,” as used herein does not necessarily refer to the same embodiment, although it may.

The present invention provides a composition suitable to withstand the requirements of textile processing temperatures, water dispersibility, compatibility with acetic acid, and low skin toxicity profiles.

In an embodiment of the invention, the composition comprises a component selected from the group consisting of benzoic acid, a salt of benzoic acid, non-benzoic acid, a salt of non-benzoic acid, and a combination thereof. The composition may further comprise an optional binder.

A non-limiting example of a salt of benzoic acid is sodium benzoate.

Non-limiting examples of non-benzoic acid are sorbic acid, citric acid, lactic acid, acetic acid, L-glutamic acid, or a combination thereof.

Non-limiting examples of a salt of a non-benzoic acid are potassium sorbate, trisodium citrate, and a combination thereof.

Preferably, the binder comprises an acrylic.

In a preferred aspect of the invention, the composition comprises a combination of sodium benzoate and a non-benzoic acid. For example, sodium benzoate and a non-benzoic acid selected from the group consisting of sorbic acid, citric acid, lactic acid, acetic acid, L-glutamic acid, and a combination thereof.

It is surprisingly found that there is a synergistic effect achieved with a combination of sodium benzoate and a non-benzoic acid in regard to odor control and durability to home laundering when applied on a textile. Sodium benzoate and non-benzoic acid are in a weight ratio in a range of 1:3 to 10:1.

Preferably, the composition comprises sodium benzoate and citric acid. The composition of sodium benzoate and citric acid are preferably in a weight ratio in a range of 1:3 to 10:1. For example, experiments conducted below illustrate a ratio of sodium benzoate to citric acid in a 1:1 ratio. The composition may be in powder form, liquid form, or a combination thereof. Preferably, the composition is in powder form but also applicable in liquid form. The components of the composition can be added as a mixture or separately, either in powder form, liquid form, or a combination thereof.

In an aspect of the invention, methods of preparation and/or using are provided. Any number of textile application or treatment methods including, but not limited to, padding or exhaustion can be used.

A method of the invention comprises applying the composition of the invention to a textile by exhaustion. For example, the method comprises adding benzoic acid (and/or a salt of the benzoic acid), or sodium benzoate and a non-benzoic acid (and/or a salt of the non-benzoic acid), and an optional binder, to an exhaustion bath; exhausting a textile such as a fabric; and drying. Exhausting preferably occurs at a temperature in a range of 85° C. to 130° C. and/or for a period of time in a range of 20 minutes to 45 minutes.

A method of the invention comprises applying the composition to a textile by padding. For example, the method comprises adding benzoic acid (and/or a salt of the benzoic acid), or sodium benzoate and a non-benzoic acid (and/or a salt of the non-benzoic acid), and an optional binder to a padding bath; padding the composition on a textile such as a fabric; and curing at 130° C. to 160° C. for 45 seconds to 1 minute.

The binder is optional and may be added depending upon the final textile washing durability requirements. Preferably, the binder is an acrylic binder. However, it was surprisingly found that inclusion of the binder enhanced the already unexpected antimicrobial benefit.

Bacteria control on a textile material can be activated and enhanced by laundry process. Advantages of the present invention the composition of the present invention is non-hazardous and is applicable to most forms of textile manufacturing.

### EXAMPLE 1

Benzoic acid with an acrylic binder was padded on 100% polyester. Antimicrobial efficacy before and after 25 home laundry (HL) washes had been tested following AATCC TM100. Bacteria used for the test is kleb pneumoniae. Data is shown in Table 1. At both 0 HL and 25 HL significant log reduction were observed, and data was repeatable in different runs.

TABLE 1

| Log | | | Log | | |
|---|---|---|---|---|---|
| Reduction | Runt | Run2 | Reduction | Runt | Run2 |
| Raw Data | (OHL) | (OHL) | Raw Data | (25H1) | (25HL) |
| | 4.7 | 4.7 | | 4.4 | 3.4 |
| | 4.7 | 4.7 | | 1.6 | 3.4 |
| | 4.7 | 4.7 | | 3.9 | 1.7 |
| Avg. | 4.7 | 4.7 | Avg. | 3.3 | 2.8 |
| STDEV | 0 | 0 | STDEV | 1.2 | 0.8 |

### EXAMPLE 2

This example illustrates exhaustion results using a composition comprising sodium benzoate and a non-benzoic acid. The non-benzoic acid used was citric acid. The composition was applied on 100% polyester. The results were obtained from a mill trial. Test conditions were 16% applied based on weight of fabric (o.w.f.) and a 1:1 ratio of sodium benzoate to citric acid. Data are shown in Table 2.

TABLE 2

| | 0 HL | | 1 HL | | 5 HL | | 25 HL | | 50 HL | |
|---|---|---|---|---|---|---|---|---|---|---|
| | Kp | Sa | Kp | Sa | Kp | Sa | Kp | Sa | Kp | Sa |
| Run 1 | 0 | 0 | 0.2 | 1 | 1.2 | 2 | 1.8 | 2.2 | 1.8 | 2.9 |
| | 0 | 0 | 0 | 1.6 | 1.4 | 3.9 | 2.2 | 4.4 | 2 | 2.9 |
| | 0 | 0 | 0.1 | 1.8 | 1.7 | 3.4 | 2.7 | 2.7 | 1.8 | 3.2 |
| Run 1 | 0 | 0 | 0.1 | 1.5 | 1.4 | 3.1 | 2.2 | 3.1 | 1.9 | 3.0 |
| Average | | | | | | | | | | |
| Run 2 | 0 | 0 | 0.8 | 0 | 1.4 | 3.4 | 1.9 | 3.9 | 1.8 | 4.7 |
| | 0 | 0 | 1.1 | 2.6 | 1.4 | 3.4 | 1.8 | 3.6 | 1.4 | 4.7 |
| | 0 | 0 | 0 | 1.9 | 1.2 | 3.5 | 2 | 4.2 | 1.8 | 4.2 |
| Run 2 | 0 | 0 | 0.6 | 1.5 | 1.3 | 3.4 | 1.9 | 3.9 | 1.7 | 4.5 |
| Average | | | | | | | | | | |

Exhaustion efficacy had been tested following AATCC TM100 on 100% polyester. No efficacy had been detected before wash. Reduction rate of both kleb pneumoniae and staphylococcus aureus were increased along with washing times.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A method for reducing microbial growth and odor on a textile material, the method comprising applying a composition to a textile material, the composition consisting of;
a salt of benzoic acid;
a non-benzoic acid selected from the group consisting of sorbic acid, citric acid, lactic acid, acetic acid, L-glutamic acid, trisodium citrate, potassium sorbate, and combinations thereof;
wherein the composition has a weight ratio of salt of benzoic acid to non-benzoic acid ranging from 1:3 to 10:1; and
the composition reduces microbial growth on the textile after laundering.

2. The method according to claim 1, wherein applying the composition comprises exhausting the composition onto the textile.

3. The method according to claim 2, wherein exhausting the composition onto the textile comprises diluting the composition in an aqueous solution.

4. The method according to claim 2, wherein exhausting occurs at a temperature in a range of 85° C. to 130° C.

5. The method according to claim 2, wherein exhausting occurs for a period of time in a range of 20 minutes to 45 minutes.

6. The method according to claim 1, wherein applying the composition comprises padding the composition onto the textile.

7. The method according to claim 6, further comprising curing the textile at 130° C. to 160° C. for 45 seconds to one minute.

8. A method for reducing microbial growth and odor on a textile material, the method comprising applying a composition to a textile material, the composition consisting of;
sodium benzoate, and
citric acid
wherein the composition has a weight ratio of sodium benzoate to citric acid ranging from 1:3 to 10:1; and
the composition reduces microbial growth on the textile after laundering.

9. The method according to claim 8, wherein applying the composition comprises exhausting the composition onto the textile.

10. The method according to claim 9, wherein exhausting the composition onto the textile comprises diluting the composition in an aqueous solution.

11. The method according to claim 9, wherein exhausting occurs at a temperature in a range of 85° C. to 130° C.

12. The method according to claim 9, wherein exhausting occurs for a period of time in a range of 20 minutes to 45 minutes.

13. The method according to claim 8, wherein applying the composition comprises padding the composition onto the textile.

14. The method according to claim 13, further comprising curing the textile at 130° C. to 160° C. for 45 seconds to one minute.
